(19) United States
(12) Patent Application Publication
(10) Pub. No.: US 2022/0143313 A1
(43) Pub. Date: May 12, 2022

(54) Clip-On Flow Control for IV Lines

(71) Applicant: Zyno Medical, LLC, Natick, MA (US)

(72) Inventors: Chao Young Lee, Weston, MA (US); Zhenhua Mao, Andover, MA (US); Mei Zhang, Sharon, MA (US)

(21) Appl. No.: 17/520,182

(22) Filed: Nov. 5, 2021

Related U.S. Application Data
(60) Provisional application No. 63/110,647, filed on Nov. 6, 2020.

(51) Int. Cl. A61M 5/168; A61M 5/14; A61M 5/142

(52) U.S. Cl. CPC A61M 5/16881; A61M 5/1418; A61M 5/142; A61M 2205/581; A61M 2205/3331; A61M 2205/0244; A61M 2205/8206

(57) ABSTRACT

A “clip-on” flow controller for IV lines automatically controls flow rate over a range of pressures through a self-contained valve and flow sensor. By eliminating a pump, a lightweight system with a long operating life can be produced, as well as a system that can be self-supported on the IV line yet still provide institutional-level flow accuracy without accurate elevation of the IV bag or careful monitoring.

### CROSS REFERENCE TO RELATED APPLICATION

This application claims the benefit of U.S. Provisional Application No. 63/110,647, filed Nov. 6, 2020, hereby incorporated by reference.

### BACKGROUND OF THE INVENTION

The present invention relates to systems for intravenous (IV) administration of drugs and, in particular, to a system allowing improved flow-control of IV bags medicines used in extra-institutional settings.

Precise delivery of IV medications to a patient may be provided by an IV pump, for example, supported on a pole stand holding an elevated IV bag. An IV line from the IV bag is threaded through the pump to communicate with a needle terminating the IV line and introduced into the patient. Operation of the IV pump (for example, a peristaltic pump) can then provide a controlled metering of the IV fluid.

Portable IV pumps that can provide similar metering are also known, for example, in the delivery of medications to a patient in an ambulatory setting. Such pumps include a battery providing power to the pump and mobility to the patient. In some cases, the pump may be sufficiently light to be worn by the patient, for example, on a belt or the like also holding the IV bag.

When IV fluids need to be delivered outside of an institutional setting, conventional gravity feed of the IV fluid may be relied upon, with the height of the IV bag adjusted to obtain a desired flow rate, for example, visually monitored by counting drops of liquid in a clear chamber attached to the IV line.

### SUMMARY OF THE INVENTION

The present invention provides a “clip-on” flow controller for IV lines that may automatically control flow rate over a range of pressures through a self-contained valve and flow sensor. By eliminating a pump, a lightweight system with a long operating life can be produced, as well as a system that can be self-supported on the IV line yet still provide institutional-level flow accuracy without accurate elevation of the IV bag or careful monitoring.

In one embodiment of the present invention, a clip-on IV line flow controller has a housing adapted to releasably receive through a central passage an IV line extending therethrough and exposed on opposite sides of the housing, the housing sized and adapted to be self-supporting on the IV line, the housing containing an electrically operable valve; a control circuit communicating with the electrically operable valve and controlling flow through the IV line as received by the housing according to a measured flow through the IV line and a desired flow set value; a user input control for entering the desired flow set value to the control circuit; a user output indicator from the control circuit indicating at least one measured flow range; and a battery providing power to the control circuit and the electrically operable valve.

It is thus a feature of at least one embodiment of the present invention to greatly improve IV delivery accuracy in situations where a standard IV pump system is not available or practical.

The housing may provide first and second housing portions releasably attachable to clamp about the IV line and be held by a catch against sliding along the IV line.

It is thus a feature of at least one embodiment of the present invention to permit the housing to be closed around an IV line to clamp with the IV line without compressing the IV line so that the housing does not slide along the IV line.

At least one edge of the first and second housing portions may support a set of attachment elements adapted to releasably attach the first and second housing portions.

It is thus a feature of at least one embodiment of the present invention to allow the flow controller to be reused for multiple drug deliveries by opening the housing and replacing the used IV line.

At least one edge of the first and second housing portions may support a hinge to allow pivotable engagement of the first and sections of the housing.

It is thus a feature of at least one embodiment of the present invention to encourage alignment of the IV line with the valve and flow detector within the housing through an easy to open and close housing.

A central tubing section of the IV line may be sized for receipt within the housing and may be positioned to communicate a volume of fluid from a first tubing section of the IV line adapted for attachment to an IV bag to the lumen of the central tubing section and the volume of fluid from the lumen of the central tubing section to a second tubing section of the IV line adapted for attachment to a patient.

It is thus a feature of at least one embodiment of the present invention to permit a specialized soft tubing to be incorporated into the housing to provide more accurate flow detection and flow control.

The central tubing section may be more flexible than a remainder of the IV line. The central tubing section may be a silicone rubber tubing.

It is thus a feature of at least one embodiment of the present invention to use resilient tubing that is not prone to breakage when compressed by the valve and that can be connected to stiffer conventional IV lines.

A flow sensor may be adapted to measure flow through the IV line and is integrated into the central tubing section.

It is thus a feature of at least one embodiment of the present invention to allow for high sensitivity detection to be built into the specialized tubing at lower manufacturing costs.

The flow sensor may be a MEMS flow sensor.

It is thus a feature of at least one embodiment of the present invention to permit detection of very low flow rates (through pressures of the IV fluid through the IV line) at low power consumption levels, and to detect air bubbles via thermally sensitive resistors.

An audio transducer may be supported by the housing and may communicate with the controller to produce an audible indication when the at least one measured flow range is below a desired flow set value and that the IV bag should be further elevated.

It is thus a feature of at least one embodiment of the present invention to ensure sufficient fluid flow through the IV line of a gravity feed system without a pump via proper elevation of the IV bag.

The control circuit may communicate with the electrically operable valve and control operation of the valve according to the measured flow through the IV line to stop flow when a desired dose amount has been delivered.

It is thus a feature of at least one embodiment of the present invention to automatically stop fluid flow following a completed drug delivery or in some instances when air bubbles are detected.

The electrically operable valve may be a pinch valve adapted to adjust an opening of the IV line by compressing the IV line.

It is thus a feature of at least one embodiment of the present invention to adjust flow through the IV line without contaminating the inner lumen of the IV line.

A set of attachment elements may be a locking feature requiring a special tool to release the locking features.

It is thus a feature of at least one embodiment of the present invention to prevent tampering to or accidental opening of the housing and may be locked or unlocked by a pharmacist prior to delivery to the patient drug delivery site.

A first tubing connector and a second tubing connector may connect first and second ends of the central tubing section with the IV line. The first tubing connector and second tubing connector may be Luer lock connectors.

It is thus a feature of at least one embodiment of the present invention to allow the specialized tubing to be adaptable with preexisting, conventional IV lines through a Luer lock connector attachment to the IV line.

The housing may be less than 4 inches in its longest dimension. The housing may be less than 6 ounces in weight.

It is thus a feature of at least one embodiment of the present invention to allow the housing to be self-supported on the IV line without distention to the IV line.

A strap may be supported by the housing and may be sized to be worn on a patient's arm.

It is thus a feature of at least one embodiment of the present invention to prevent pullout of the IV line when the end of the IV line includes a needle inserted into the patient.

The housing may be exclusive of a pump.

It is thus a feature of at least one embodiment of the present invention to provide low cost, low energy consumption hardware elements that can be disposed of after a predetermined amount of use.

In one embodiment of the present invention, a method of controlling fluid flow through an IV line includes providing a clip-on IV line flow controller comprising a housing adapted to releasably receive through a central passage an IV line extending therethrough and exposed on opposite sides of the housing, the housing sized and adapted to be self-supporting on the IV line, the housing containing an electrically operable valve; a control circuit communicating with the electrically operable valve and controlling flow through the IV line as received by the housing according to a measured flow through the IV line and a desired flow set value; a user input control for entering the desired flow set value to the control circuit; a user output indicator from the control circuit indicating at least one measured flow range; and a battery providing power to the control circuit and the electrically operable valve; and controlling flow through the IV line as received by the housing according to a measured flow through the IV line and a desired flow set value.

In one embodiment of the present invention medical tubing includes a central tubing section less than 12 inches in length; a first tubing connector attached to a first end of the central tubing section and adapted to attach a first branch tubing section adapted for attachment to an IV bag to communicate a volume of fluid of the IV bag to the lumen of the central tubing section; a second tubing connector attached to a second end of the central tubing section and adapted to attach a second branch tubing section to communicate the volume of fluid from the lumen of the central tubing section to the lumen of the second branch tubing section; and a MEMS flow sensor integrated into the central tubing section and adapted to sense a flow of fluid through the central tubing section.

These particular objects and advantages may apply to only some embodiments falling within the claims and thus do not define the scope of the invention.

### BRIEF DESCRIPTION OF THE DRAWINGS

FIG. 1 is a perspective view of the clip-on flow controller of the present invention attached to a short length of specially prepared IV tubing that may be clipped into a longer IV line system;

FIG. 2 is a fragmentary top plan view of the flow controller of FIG. 1, opened to show an internal mechanism;

FIG. 3 is a fragmentary perspective view of the short length of specially prepared IV tubing having a MEMS (Micro Electro Mechanical System) flow sensor embedded therein; and

FIG. 4 is a fragmentary perspective view of a strap that may be associated with the flow controller of FIG. 1 for attachment of the flow controller to the patient.

### DETAILED DESCRIPTION OF THE PREFERRED EMBODIMENT

### General Description

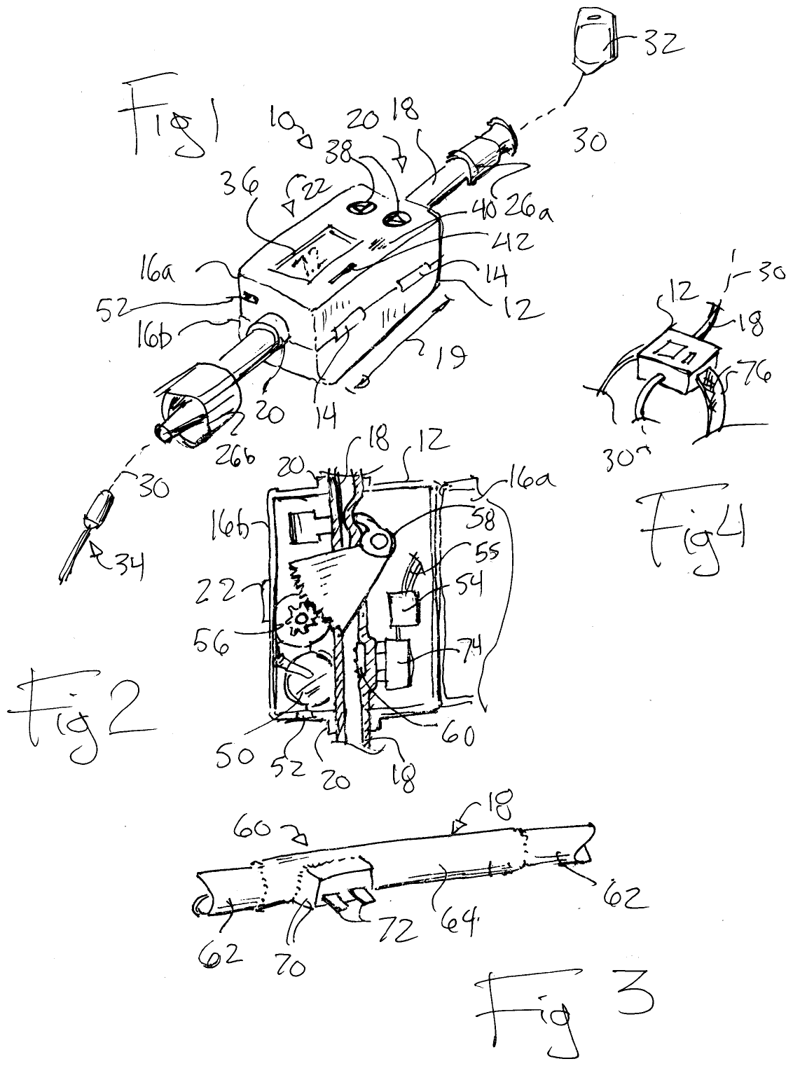

Referring now to FIG. 1, a clip-on flow controller 10 per the present invention may provide for a rigid outer housing 12, for example, constructed of injection molded thermoplastic having hinges 14, for example, living hinges, on one side allowing the housing 12 to separate into opposed housing halves 16a and 16b for receipt of a short length of IV tubing 18 and then to be closed to retain the IV tubing 18 as it passes through the housing 12 and out opposite ends of the housing 12.

Each end of the housing 12 through which the IV tubing 18 passes, may provide for a collar separating into halves 16 and assembling around the IV tubing 18 when the halves are closed to clamp the IV tubing 18 without crushing or closing the IV tube lumen yet so that the housing 12 is retained on the IV tubing 18 without slippage therealong, the weight of the housing 12 and its contained components fully supported by the IV tubing 18.

The halves 16 may be held in the closed state against the resilience of the IV tubing 18 by a latch 22 (not visible in FIG. 1) providing a locking feature, for example, requiring a special tool to release the latch 22 once it is closed to prevent accidental release.

The ends of the IV tubing may have standard Luer locks 26a and 26b allowing the ends to be spliced or clipped into a longer IV line 30 having similar connectors, for example, leading from an IV bag 32 elevated for gravity flow at one end and to a needle set 34 at the other end.

An upper face of the upper half 16a of the housing 12 exposes an LCD display 36 indicating a flow rate and maximum desired drug delivery amount (for example, alternating between these two values). Membrane buttons 38 positioned near the display 36 allow for adjustment of the flow rate and maximum flow amount through the IV tubing 18 as will be discussed. A piezoelectric audio transducer 40 provides audible tones, for example, warning if the housing halves 16a and 16b are not fully closed or if flow rate stops or drops below a predetermined lower limit, for example, caused by kinking or occlusion in the IV line 30. As will be discussed the flow rate sensor may also detect air bubbles. In addition, an on off switch 42 allows the flow controller 10 to be turned off for storage or the like. An optional charging port 52 may also be provided as will be discussed below.

Generally, the clip-on flow controller 10 will be both small and of light weight, for example, having a longest dimension 19 less than 4 inches and desirably less than 3 inches and a total weight of less than 8 ounces and desirably less than 6 ounces to be easily supported by the IV line 18 and 30 and the Luer locks 26 without undue distortion of or damage to the IV lines 18, 30 or damage to the IV bag 32 or its hanger.

Referring now to FIG. 2, in one embodiment, housing 12 may hold a long life battery, for example, having a 10-year nominal rating such as non-rechargeable lithium thional chloride 3.6 volt cell, making the flow controller 10 suitable for long-term storage and use in emergency kits or the like. Alternatively, standard lithium-ion rechargeable cells or other similar batteries may be used that may be recharged through the charging port 52 discussed above.

The battery 50 may provide power to a controller 54 (through power switch 42 shown in FIG. 1) controlling a motor 56 and providing communication to the display 36, buttons 38, and audio transducer 40 per interconnect wiring 55. The motor 56 operates a pinch valve 58 squeezing a portion of the IV tubing 18 to control the flow therethrough. This operation of the motor 56 and pinch valve 58 may be according to a feedback loop using a flow sensor 60 built into the IV tubing 18. Under a range of different pressures of IV fluid in the IV tubing 18, for example, proportional to a variety of different elevations of the IV bag 32, the motor 56 may adjust the pinch valve 58 to provide a steady flow rate at a set point of the feedback loop according to the measured flow through the IV tubing 18 by the flow sensor 60.

Referring also to FIG. 3, the IV tubing 18 may include two end-most stiff vinyl tubing portions 62 attaching each to the Luer locks 26a and 26b and joined respectively to ends of a central softer silicone rubber section 64 suitable for the pinch valve 58.

The flow sensor 60 may preferably be a MEMS (Micro Electro Mechanical System) type flow sensor, for example, commercially available from Sensirion of Switzerland under the trade name LD20 or other similar device making use of thermal time-of-flight technologies or other similar highly sensitive flow rate measurement systems. This technology may not only measure extremely low flow rates accurately but may detect air bubbles based on their different thermal characteristics. The active membrane of this sensor providing thermally sensitive resistors may be exposed to the central lumen of the IV tubing 18. Electrical signals from this membrane attached to conductors provide sealed passage through an overmolding 70 to exposed electrical connectors 72 that may be received by a connector block 74 attached to the housing 12 communicating with the controller 54.

Referring now to FIG. 4, while the housing 12 may be fully self-supporting on the IV tubing 18 and 30, a strap 76 may be added to the housing 12 in the manner of a wristwatch strap or band to allow the flow controller 10 to be stabilized, for example, by strapping it to a patient's arm or the like and thereby preventing pullout of the IV line. Self-supporting in this context means that the housing can be indefinitely supported on the IV line, for example the latter suspended vertically for example from an IV bag, without undue distention of the IV line or damage there to.

In use, the flow controller 10 may be quickly installed on an IV line 30 either using an IV line 30 integrated with a silicone rubber section 64 and in molded to sensor 60 or using the short IV tubing 18 depicted above and attached via the Luer locks 26. The flow controller 10 may be switched on, triggering a self-check confirming operation of the pinch valve 58, for example, by monitoring excess current or a limit switched as the motor moves the pinch valve between extreme positions to determine its initial starting position. A flow rate may be preloaded and the flow controller 10 may begin operation immediately, for example, in emergency applications. Otherwise, the membrane buttons 38 may be used to set a desired flow rate. The IV bag 32 is then elevated until a tone from the audio transducer 40 indicates that sufficient flow rate has been obtained. Normally the user will be instructed to add slight additional elevation of the IV bag 32 to ensure that the control range of the flow controller 10 may be satisfied and no further adjustment is necessary.

During infusion, flow is monitored and controlled and warning tones are produced if flow is blocked or obstructed or an air bubble is detected (upon which the pinch valve closes).

If a total desired dose amount has been entered by the user, at the conclusion of that dose delivery (obtained by integrating the flow rate) the pinch valve closes and a signal is provided to the user.

At the conclusion of the infusion, the IV tubing 18 may be removed and replaced with a new IV line that has sterile properties and the previously used IV tubing 18 is disposed. The controller 54 may track the battery reserve, for example, by counting the number of motor cycles, battery age, recharges, etc. to ensure ample capacity exists for the next infusion.

In at least some embodiments, the controller 54 may check the battery reserve prior to every new drug delivery operation or motor 56 and flow sensor 60 operation to determine if there is sufficient battery reserve or battery life remaining for a typical drug delivery. The controller may check for adequate remaining battery energy for the drug delivery using an internal time-to-ampere-hour conversions based on the current drain of the controller 54, motor 56 and flow sensor 60 and compare that against the estimated total ampere-hours of the battery 50. If the battery reserve is less that what would be needed, the controller 54 may indicate to the user that the battery 50 should be charged or replaced prior to operation. The controller 54 may not permit operation of the motor 56 and flow sensor 60 until the battery 50 is charged to a sufficient level to prevent unwanted interruptions during delivery.

In at least some embodiments, the number of uses of the flow controller 10 may be counted, for example, determined by accumulating the number of IV tubing replacements, motor cycles, battery life, recharges, and other lifespan tracking methods, in order to alert the user or automatically shut off when the flow controller 10 has reached its intended service life limits, should no longer be used, and should be disposed. Therefore, it is understood that the flow controller 10 may be intended as a disposable use device with limited service life.

By eliminating the pump, extremely lightweight and compact size may be obtained. Desirably the pinch valve 58 may be constructed so that once its position has been set, no power is necessary to hold IV tubing 18 in the desired compressed configuration, for example, through a general resistance of the reverse mechanical advantage of that gear train or through other mechanisms to reduce electrical power usage to a minimum. It will be appreciated that other valve designs may also be used, for example, those which provide for a rotating stopcock or the like.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. A clip-on IV line flow controller comprising:
a housing adapted to releasably receive through a central passage an IV line extending therethrough and exposed on opposite sides of the housing, the housing sized and adapted to be self-supporting on the IV line, the housing containing:
an electrically operable valve;
a control circuit communicating with the electrically operable valve and controlling flow through the IV line as received by the housing according to a measured flow through the IV line and a desired flow set value;
a user input control for entering the desired flow set value to the control circuit;
a user output indicator from the control circuit indicating at least one measured flow range; and
a battery providing power to the control circuit and the electrically operable valve.

2. The flow controller of claim 1 wherein the housing provides first and second housing portions releasably attachable to clamp about the IV line and be held by a catch against sliding along the IV line.

3. The flow controller of claim 2 wherein at least one edge of the first and second housing portions supports a set of attachment elements adapted to releasably attach the first and second housing portions.

4. The flow controller of claim 2 wherein at least one edge of the first and second housing portions supports a hinge to allow pivotable engagement of the first and sections of the housing.

5. The flow controller of claim 1 further comprising a central tubing section of the IV line sized for receipt within the housing and positioned to communicate a volume of fluid from a first tubing section of the IV line adapted for attachment to an IV bag to a lumen of the central tubing section and the volume of fluid from the lumen of the central tubing section to a second tubing section of the IV line adapted for attachment to a patient.

6. The flow controller of claim 5 wherein the central tubing section is more flexible than a remainder of the IV line.

7. The flow controller of claim 6 wherein the central tubing section is a silicone rubber tubing.

8. The flow controller of claim 5 wherein a flow sensor adapted to measure flow through the IV line is integrated into the central tubing section.

9. The flow controller of claim 8 wherein the flow sensor is a MEMS flow sensor.

10. The flow controller of claim 1 further including an audio transducer supported by the housing and communicating with the controller to produce an audible indication when the at least one measured flow range is below a desired flow set value and that the IV bag should be further elevated.

11. The flow controller of claim 1 wherein the control circuit communicates with the electrically operable valve and control operation of the electrically operable valve according to the measured flow through the IV line to stop flow when a desired dose amount has been delivered.

12. The flow controller of claim 1 wherein the electrically operable valve is a pinch valve adapted to adjust an opening of the IV line by compressing the IV line.

13. The flow controller of claim 3 wherein the set of attachment elements is a locking feature requiring a special tool to release the locking features.

14. The flow controller of claim 5 further comprising a first tubing connector and a second tubing connector connecting first and second ends of the central tubing section with the IV line

15. The flow controller of claim 14 wherein the first and second tubing connectors are Luer lock connectors.

16. The flow controller of claim 1 wherein the housing is less than 4 inches in its longest dimension.

17. The flow controller of claim 1 wherein the housing is less than 6 ounces in weight.

18. The flow controller of claim 1 further comprising a strap supported by the housing and sized to be worn on a patient's arm.

19. The flow controller of claim 1 wherein the housing is exclusive of a pump.

20. A method of controlling fluid flow through an IV line comprising:
providing a clip-on IV line flow controller comprising:
a housing adapted to releasably receive through a central passage an IV line extending therethrough and exposed on opposite sides of the housing, the housing sized and adapted to be self-supporting on the IV line, the housing containing:
an electrically operable valve;
a control circuit communicating with the electrically operable valve and controlling flow through the IV line as received by the housing according to a measured flow through the IV line and a desired flow set value;
a user input control for entering the desired flow set value to the control circuit;
a user output indicator from the control circuit indicating at least one measured flow range; and
a battery providing power to the control circuit and the electrically operable valve; and
controlling flow through the IV line as received by the housing according to a measured flow through the IV line and a desired flow set value.

21. A medical tubing comprising:
a central tubing section less than 12 inches in length;
a first tubing connector attached to a first end of the central tubing section and adapted to attach a first branch tubing section adapted for attachment to an IV bag to communicate a volume of fluid of the IV bag to a lumen of the central tubing section;
a second tubing connector attached to a second end of the central tubing section and adapted to attach a second branch tubing section to communicate the volume of fluid from the lumen of the central tubing section to a lumen of the second branch tubing section; and
a MEMS flow sensor integrated into the central tubing section and adapted to sense a flow of fluid through the central tubing section.
